(19) United States
(12) Patent Application Publication
(10) Pub. No.: US 2025/0082494 A1
(43) Pub. Date: Mar. 13, 2025

(54) ReliEVE (Name). Electric Portable, Male organ-like device for women

(71) Applicant: Francisco Diaz Solis, Little Falls, NJ (US)

(72) Inventor: Francisco Diaz Solis, Little Falls, NJ (US)

(21) Appl. No.: 18/367,040

(22) Filed: Sep. 12, 2023

(51) Int. Cl. A61F 5/455; A61F 5/44

(52) U.S. Cl. CPC A61F 5/4556; A61F 5/4404

(57) ABSTRACT

The present invention discloses a portable male organ-like device, named “ReliEVE,” designed for women to urinate while standing. The device comprises various components, including a fluid collector, a pump, a rechargeable battery, and a moldable end, enabling women to experience the convenience and functionality associated with male urination. The invention offers a hygienic and efficient alternative for women, addressing the limitations of conventional bathroom facilities. The device provides control over the urine stream, ensuring a comfortable and practical experience.

### BACKGROUND OF THE INVENTION

The present invention relates to an electric portable male organ-like device designed for women to address the challenge of urinating while standing. This invention provides a novel solution to the limitations faced by women when using conventional bathroom facilities, offering a hygienic, convenient, and comfortable alternative.

### FIELD OF THE INVENTION

The invention generally pertains to the field of personal hygiene devices. More specifically, it relates to devices that enable women to urinate while standing, simulating the functionality and convenience of a male organ.

### BRIEF SUMMARY OF THE INVENTION

The present invention, named “ReliEVE,” is an electric portable male organ-like device specifically designed to allow women to urinate while standing. It comprises several components, including a fluid collector or plastic receptacle, a fine netting screen, a plastic pump with motor and electric wires, a motor board, a rechargeable battery, a plastic hose, a moldable plastic end, and a push on/release off button. These components work together to provide a convenient and effective solution for female urination in a standing position.

### BRIEF DESCRIPTION OF THE DRAWINGS

FIG. 1 depicts a cutaway view of the electric portable, male organ-like device for women.

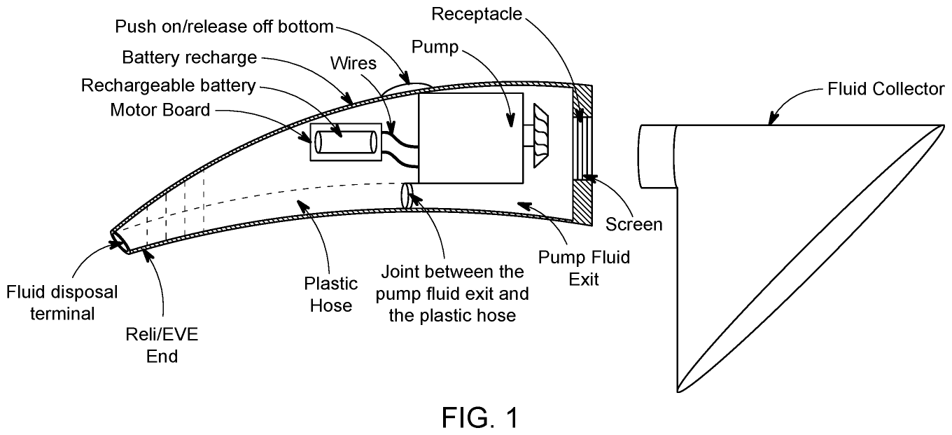

FIG. 2 depicts a cutaway view of the electric portable, male organ-like device for women.

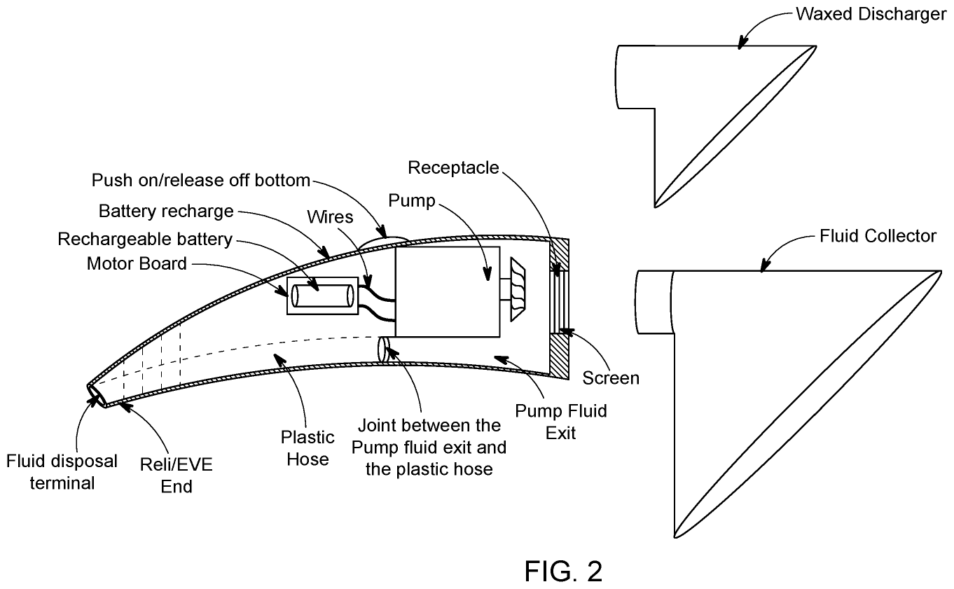

### DETAILED DESCRIPTION OF THE INVENTION

The following detailed description of the invention provides a comprehensive overview of the ReliEVE device and its functionalities, components, and operational characteristics.

The invention comprises the following elements, described from one end to the other: Fluid Collector or Plastic Receptacle, Fine Netting Screen, Plastic Pump with Motor and Electric Wires, Motor Board, Rechargeable Battery, Plastic Hose, Moldable Plastic End, and Push On/Release Off Button.

The device features a fluid collector or plastic receptacle with an anatomically adapted shape to fit securely around the vulva and its adjacent area, known as the perineal area, in order to collect efficiently the urine without leaking. The receptacle narrows into a funnel-like structure towards the lower area.

Inside the receptacle, a middle section is designed to accommodate a perforated waxed cup, providing a disposable and convenient option for fluid collection.

Positioned at the end of the funnel, a fine netting screen serves to filter out unwanted particles during the urination process, ensuring cleanliness and preventing blockages.

The device includes a plastic pump equipped with a motor, fan, and electric wires. These wires are connected to a motor board, which serves as the control unit for the device's electrical components.

The motor board regulates the functioning of the pump and coordinates the operation of the electrical components of the device.

The ReliEVE device incorporates a rechargeable battery, providing a portable and reliable power source for extended usage. The battery can be conveniently recharged through a designated hole on the device.

A plastic hose is internally connected to the pump, conducting the fluid from the pump to the opposite end of the device. This ensures a seamless transfer of urine without leakage or spillage. The plastic hose functions as a ureter, allowing a flow of urine from the plastic pump to where the urine is subsequently expelled.

At the opposite end of the plastic hose, a moldable plastic end is provided. This end allows for precise direction and redirection of the fluid stream, simulating the functionality and control associated with using a natural male organ.

Positioned at a convenient location for the user, a push on/release off button provides control over the device's operation, allowing the user to initiate and cease the flow of urine as desired. It stops the pump from running after being used which would otherwise damage it. Furthermore, it cleans the main ReliEVE components after use.

1. An electric portable male organ-like device for women to facilitate urination while standing, comprising:
a. A fluid collector or plastic receptacle shaped to be adapted to the vulva, with a funnel-shaped lower area;
b. A fine netting screen positioned at the end of the funnel;
c. A plastic pump with a motor, fan, and electric wires, the electric wires connecting to a motor board;
d. A motor board serving as a control unit for the device's electrical components;
e. A rechargeable battery providing power for the device;
f. A plastic hose internally conducting the fluid from the pump to the opposite end;
g. A moldable plastic end at the opposite end of the hose, allowing direction and redirection of the fluid stream;
h. A push on/release off button positioned for user convenience; and
i. A rechargeable battery hole for recharging the battery.

2. The device of claim 1, further comprising an adaptation within the receptacle to hold a perforated waxed cup for disposable fluid collection.

3. A method for allowing women to urinate while standing, comprising the steps of:
a. Providing an electric portable male organ-like device as described in claim 1;
b. Positioning the device for optimal fit around the vulva;
c. Initiating the flow of urine through the push on/release off button; and
d. Directing and redirecting the urine stream using the moldable plastic end.

4. The method of claim 3, further comprising the option of using a disposable perforated waxed cup within the device's receptacle for fluid collection.
